(19) United States
(12) Patent Application Publication
(10) Pub. No.: US 2020/0051687 A1
(43) Pub. Date: Feb. 13, 2020

(54) CAPTURING AND UTILIZING RESOURCE USAGE FOR A CLINICAL PATHWAY

(71) Applicant: KONINKLIJKE PHILIPS N.V, EINDHOVEN (NL)

(72) Inventors: Andre Melon BARROSO, Aachen (DE); Ana Jorge RODRIGUES DE MOURA LEITAO, Eindhoven (NL)

(21) Appl. No.: 16/339,751

(22) PCT Filed: Oct. 24, 2017
(86) PCT No.: PCT/EP2017/077149

§ 371 Date: Apr. 5, 2019

Related U.S. Application Data
(60) Provisional application No. 62/411,794, filed on Oct. 24, 2016.

(51) Int. Cl. G16H 40/20; G16H 15/00; G06N 7/00

(52) U.S. Cl. CPC G16H 40/20; G06N 7/005; G16H 15/00

(57) ABSTRACT

A system (202) includes a processor (204) configured to: display, with an output device (224), a clinical pathway for a subject, wherein the clinical pathway is in an electronic document with updatable fields; receive, with an input device (222), a first signal which selects a task of the clinical pathway to complete, wherein the task is not completed; receive, with the input device, a second signal that which indicates the task is completed, wherein the task is completed; employ, with a processor (204) a probabilistic algorithm, which computes a probability value for the task; present, with the processor, a challenge question only in response to the probability value satisfying a predetermined threshold; receive, with the input device, a third signal that includes an answer to the challenge question; and update, with the processor, an updatable in the electronic document to indicate the task of the clinical pathway is completed in response to receiving the answer.

### FIELD OF THE INVENTION

The following generally relates to a system and/or method of capturing and utilizing information on resource usage for a clinical pathway.

### BACKGROUND OF THE INVENTION

A Clinical Pathway Information System (CPIS) is software executed by a computer and assists healthcare professionals to follow clinical guidelines by giving step by step guidance. The detail of the guidance depends on the connection with the Electronic Medical Record (EMR) of the healthcare institution. A clinical pathway of the CPIS aims to enhance the quality of care across the care continuum, promote patient safety, increase patient satisfaction, and optimize the use of resources.

FIG. 1 shows an example of an unspecified clinical pathway. The illustrated example clinical pathway includes a beginning and an end, and various tasks to complete in between. Since clinical pathways are prescriptive processes, workflow management systems must find a way to trigger, distribute and track tasks related to these processes. At any given instant in time, the system may generate multiple tasks, which are then queued for execution upon the availability of resources.

As hospitals serve hundreds of patients with a limited number of professionals and equipment to execute clinical tasks, the question arises on how such tasks should be prioritized to make optimal use of available resources. In process-oriented workflow systems, there is a trade-off between granularity of information monitored and burden to users; the more detailed a clinical pathway is, the more users must interact with it to feed information. Consequently, details on the actual use of resources during a task are usually not registered in the system.

Manual entry of resource utilization for each task may represent a burden to users. However, not having this information denies valuable data to process optimization algorithms, e.g., simultaneously prioritizing execution of tasks that demand the same resources creates a bottleneck in the workflow. Task scheduling algorithms rely on information about resource usage to make optimal decisions. Unfortunately, if the resource utilization information is inaccurate, such systems may produce less than desirable results.

### SUMMARY OF THE INVENTION

Aspects of the present application address the above-referenced matters and others.

According to one aspect, a system includes a processor and a memory device configured to store instructions. The processor executes the instructions, which causes the processor to: configured to: display, with an output device, a clinical pathway for a subject, wherein the clinical pathway is in an electronic document with updatable fields; receive, with an input device, a first signal which selects a task of the clinical pathway to complete, wherein the task is not completed; receive, with the input device, a second signal that which indicates the task is completed, wherein the task is completed; employ, with a processor a probabilistic algorithm, which computes a probability value for the task; present, with the processor, a challenge question only in response to the probability value satisfying a predetermined threshold; receive, with the input device, a third signal that includes an answer to the challenge question; and update, with the processor, an updatable in the electronic document to indicate the task of the clinical pathway is completed in response to receiving the answer.

In another aspect, a method, includes: displaying, with an output device, a clinical pathway for a subject, wherein the clinical pathway is in an electronic document with updatable fields; receiving, with an input device, a first signal which selects a first task of the clinical pathway to complete, wherein the first task is not completed; receiving, with the input device, a second signal that which indicates the first task is completed, wherein the first task is completed; employing, with a processor a probabilistic algorithm, which computes a first probability value for the first task; presenting, with the processor, a first challenge question only in response to the first probability value satisfying a first predetermined threshold; receiving, with the input device, a third signal that includes a first answer to the first challenge question; storing the first answer to the first challenge question in a memory; updating, with the processor, a first updatable in the electronic document to indicate the first task of the clinical pathway is completed in response to receiving the answer; analyzing the first answer along with previously received answers with an answer analysis algorithm to determine resource usage for each clinical pathway task; and employing a task scheduler to schedule tasks of clinical pathways based on the determine resource usage.

In another aspect, a non-transitory computer readable medium is encoded with computer executable instructions, which, when executed by a processor of a computer, cause the computer to: display a clinical pathway for a subject, wherein the clinical pathway is in an electronic document with updatable fields; receive a first signal which selects a task of the clinical pathway to complete, wherein the task is not completed; receive a second signal that which indicates the task is completed, wherein the task is completed; employ a probabilistic algorithm, which computes a probability value for the task; present a challenge question only in response to the probability value satisfying a predetermined threshold; receive a third signal that includes an answer to the challenge question; store the answer to the challenge question in a memory; update an updatable in the electronic document to indicate the task of the clinical pathway is completed in response to receiving the answer; analyze the answer along with previously received answers with an answer analysis algorithm to determine resource usage for each clinical pathway task; and employ a task scheduler to schedule tasks of clinical pathways based on the determine resource usage.

Still, further aspects of the present invention will be appreciated to those of ordinary skill in the art upon reading and understand the following detailed description.

### BRIEF DESCRIPTION OF THE DRAWINGS

The invention may take form in various components and arrangements of components, and in various steps and arrangements of steps. The drawings are only for purposes of illustrating the preferred embodiments and are not to be construed as limiting the invention.

FIG. 1 shows an example of a clinical pathway.

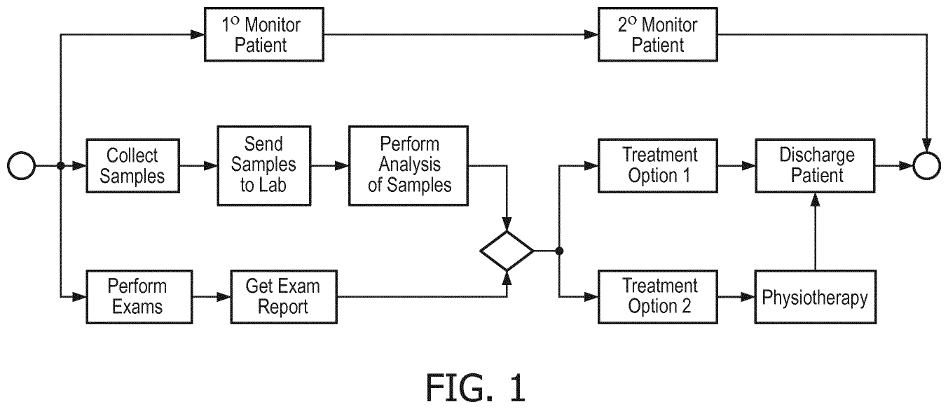

FIG. 2 schematically illustrates an example system for capturing and analyzing information on resource usage in a clinical pathway of a CPIS.

FIG. 3 schematically illustrates a method for capturing information on resource usage in a clinical pathway, where a challenge question is answered at the time the CPIS is updated to reflect a task has been completed.

FIG. 4 schematically illustrates a method for capturing information on resource usage in a clinical pathway, where a challenge question is answered within a predetermined time period after completion of a task, but not at the time the CPIS is updated to reflect the task has been completed.

FIG. 5 schematically illustrates a method for capturing information on resource usage in a clinical pathway, where a challenge question is not answered within a predetermined time period after the CPIS is updated to reflect the task has been completed.

### DETAILED DESCRIPTION OF EMBODIMENTS

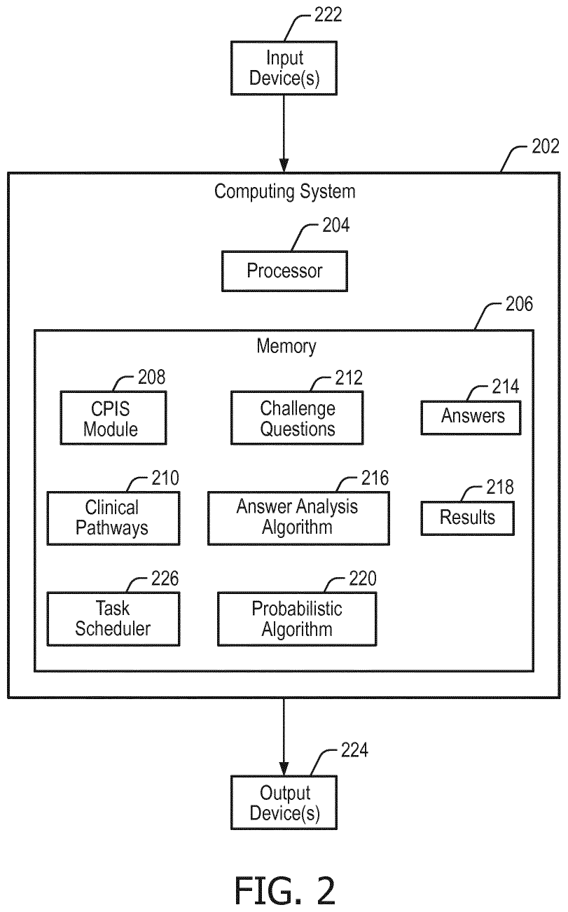

FIG. 2 illustrates an example computing system 202 configured to capture and analyze information on resource usage in a clinical pathway of a CPIS. For explanatory purposes and sake of brevity, the components are described herein as part of a single computing system (i.e., the computing system 202). However, it is understood that one or more of the components can be remote from the computing system 200 such as in the “cloud,” part of another computing system connected through a network or the like, on a server, distributed across systems, etc.

The illustrated computing system 202 includes at least one processor 204 (e.g., a microprocessor, a central processing unit, a controller, etc.) that executes at least one computer readable instruction stored in a computer readable storage medium (“memory”) 206, which excludes transitory medium and includes non-transitory medium such as physical memory and/or other non-transitory medium. The computing system 202 also includes an input device(s) 222, such as a mouse, keyboard, a touchscreen, etc., and an output device(s) 224, such as a display monitor, a touchscreen display monitor, etc.

The memory 206 stores a CPIS module 208 and clinical pathways 210. The CPIS module 208 includes an interactive graphical user interface (GUI) which has features and functionality beyond a digital representation of a paper guideline. The CPIS module 208 further includes a way to detect where a user is in the pathway (i.e. which step) either declared by the user or automatically, and optimization of management of resources in the creation of task lists for the user(s). The memory 206 also stores challenge questions 212, answers 214 to challenge questions 212, an answer analysis algorithm 216, results 218 of the answer analysis algorithm 216, and a probabilistic algorithm 220, which determines whether a challenge question 212 is presented.

The challenge questions 212 are based on criterion from a group consisting of at least one of the following: 1) Task ID; 2) Role ID; 3) Time of User (ToU); 4) Resource ID; 5) Time of Resource (ToR), and 6) Amount of Resource (AoR). The Task ID identifies the task of a clinical protocol. The Role ID identifies the type of professional responsible for execution, e.g. nurse. The ToU includes information about the time required of the user for task execution. The Resource ID identifies the type of resource used during the task e.g. MRI machine, needles or other professional. The ToR includes information about the time required of the resource during the task. The AoR includes information about the amount of the same resource that was used for completing the task.

The probabilistic algorithm 220 computes a probability P. The probability P is used to determine whether to present a challenge question when a status of a task is being changed from, e.g., “open” to “closed” via an input from the input device(s) 222. In one instance, the probabilistic algorithm 220 includes random number generator. With this algorithm, beginning with a random or a specified seed (or number), the probabilistic algorithm 220 generates a random number X that follows a probability distribution. The random number X is compared with a predetermined threshold value. If the random number X exceeds (or is equal to) the threshold value, the processor 204 does not present a challenge question. If the random number X is equal to or below (or only below) the threshold value, the processor 204 presents a challenge question.

In another instance, the probabilistic algorithm 220 employs a more complex algorithm. For example, the probabilistic algorithm 220 takes into consideration criterion from a group consisting of one or more of the following to compute the probability P: 1) the Task ID; 2) the Role ID; 3) a Frequency of User Input (FoUI); 4) a Frequency of User Access (FoUA); 5) Time to Last Input (TLI); 5) “FAIL” Challenges by User (FCU); 6) Total Amount of Challenges (TAC); 7) “DONE” Tasks by User (DTU); 8) Number of Users in a Task (NoUT); 9) Time of Challenge (ToC); 10) Average Time of Tasks (AToT); 11) Average Time of Access (AToA); 12) Task Critical Indicator (TCI); 13) Next Task Critical Indicator (NTCI), and/or other information.

An FoUI is the frequency with which each user provides input. An FoUA is the frequency with which the user accesses the CPIS module 208. A TLI is the time that has passed since the last input. An FCU is the total amount of challenges a certain user fails to respond to. A TAC is the total amount of challenges presented to a certain user. A DTU is the total amount of tasks declared by a certain user in the system. A NoUT is a number of users associated to the same Task ID. A ToC is the time the user spends completing the challenge. An AToT is an average time the user spends completing a normal task. An AToA is an average time the user spends per access to the CPIS. A TCI is a value indicating how critical the task is e.g. 0—not critical, and 1—extremely critical to be completed as soon as possible. A NTCI is a value indication of criticalness of the next task in the pathway.

In one instance, the particular combination of the above parameters takes in consideration one or more factors from a group consisting of: 1) a user is not presented with a challenge every time they use the CPIS module 208; 2) the challenges are not presented all the time to the same user; 3) there is a variety of users responding to the challenges; 4) the challenge is presented to more than one user in tasks where there are more than one professional involved, but not presented to all users in case there are more than two users in play (NoUT); 5) the user does not spend more time completing the task then a normal AToT and/or AToA; 6) a user with FCU close to TAC and/or DTU should not be presented with challenges as often as other users, and 7) a user is not presented with a challenge in case the NTCI exceeds a predetermined threshold and/or is higher than TCI by a predetermined amount.

As an example, to ensure criteria 1-3 are respected, the algorithm may simply generate for each task a random number between 0 and 1 from a uniform distribution and present the challenge question if the number is less than P. Criteria 4 can be observed by further randomly selecting a subset of users involved in the task to respond to the challenge.

For explanatory purposes, the following describes an example for gathering information of a single resource per task—the working time of the healthcare professional responsible for executing it. It is to be understood that the approach described herein is not limited to working time and/or a single resource per task, and is extendable to encompass different and/or multiple resources. The following begins with the CPIS module 208 executing and presenting a clinical pathway, where a resource to complete a task has just been utilized to complete the task.

When a user attempts to change the status of the task to “completed” via the input device(s) 222, the CPIS module 208 invokes the probabilistic algorithm 220 to decide with probability P whether a challenge question will be presented. Where the challenge question is to be presented (e.g.,), the CPIS module 208 identifies an appropriate challenge question and presents the challenge question 212 via the output device(s) 224. In this example, the challenge question 212 requests entry of an amount of time it took to execute the task. This information is entered via the input device(s) 222 and stored as part of the answers 214. In one instance, this information is indexed in the memory 206 by the task ID. In a variation, the information is otherwise indexed, and not indexed.

The challenge question is selected based on the criticality of specific resources to the task completed and the amount of information already gathered from previous challenges. The criticality resources can be added to the system by experts, while the amount of information already presented can be assessed by the number of previous answers. Challenges related to more critical resources are presented more often. Challenges within a same category of criticality are presented in proportion to the number of answers collected in a given period of time.

The answer analysis algorithm 216 processes this data, along with previously stored answers from previously presented challenge questions 212 to generate statistics, e.g. mean, medium, mode, standard deviation, etc. on the use of professional time for each task. A task scheduler 226 accesses this information and makes resource allocation decisions based thereon. A non-limiting advantage of this approach is that all users are not required to enter the aforementioned information each time they perform a task. Their burden is determined by probability P, which is chosen to balance user overhead and accuracy of the metrics computed. As the accuracy of metrics depends on the variance of responses, the ideal balance between accuracy and overhead will be assessed on a task-by-task basis. The probabilistic polling of different users over time builds information on each task at a very low burden to each user.

In a variation, the CPIS module 208 challenges a user to select from a list all resources effectively used during the task. For each resource selected, the CPIS module 208 further challenges the user for precise usage duration and/or amount of usage. Through the regular inquiry of this information, the CPIS module 208 builds a comprehensive view of resource requirements per task. A resource can also be another person, which allows to identify tasks that require more than one professional to be completed.

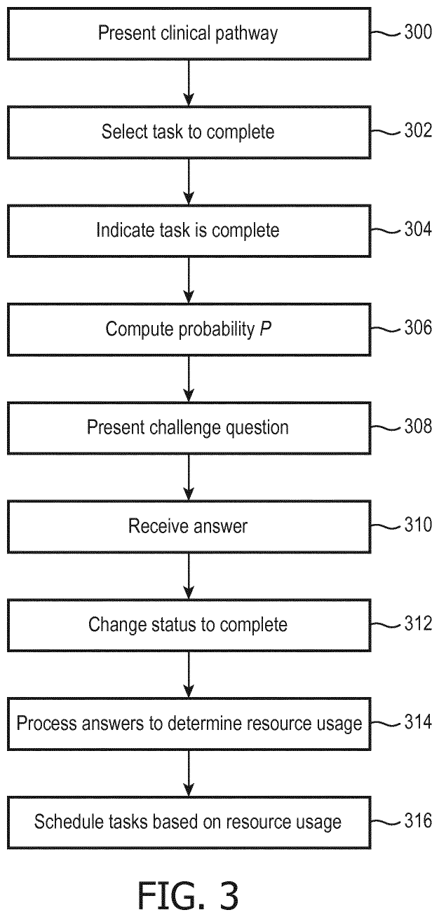

FIG. 3 schematically illustrates a method for capturing information on resource usage in a clinical pathway, where a challenge question is answered at the time the pathway is updated to reflect a task has been completed.

It is to be appreciated that the following is not limiting. That is, one or more acts can be added and/or omitted. Further, an act can occur in a different ordering.

At 300, the computing system 202 executes the instructions of the CPIS module 208, which causes the processor 204 to present at least one clinical pathway of a patient via the output device(s) 224.

In this example, the presented clinical pathway includes multiple tasks, including at least one task identified as a task to be completed.

At 302, the computing system 202, via the input device(s) 222, receives a first signal which selects the clinical task to complete.

At 304, the computing system 202, via the input device(s) 222, receives a second signal which indicates the clinical task has been completed.

At 306, the processor 204 employs the probabilistic algorithm 220 to determine whether and which challenge question will be presented, as disclosed herein and/or otherwise.

At 308, if it is determined that a challenge question will be presented, the processor 204 presents, via the output device(s) 224, a challenge question.

At 310, the computing system 202, via the input device(s) 222, receives a third signal which includes an answer to the challenge question.

At 312, the processor 204, in response to receiving the third signal, changes the status of the task in the clinical pathways 210 to indicate the task is completed.

At 314, the processor 204 stores the answer to the challenge question in the answers 214.

At 316, the processor 204 utilizes the stored answer along with other stored answers for other tasks, clinical pathways, users, etc. to determine resource usage for each task.

At 318, the processor 204 employs the answer analysis algorithm to analyze the resource usage, as disclosed herein and/or otherwise.

At 320, the processor 204 invokes the task scheduler 226 schedule tasks based on the analysis, as disclosed herein and/or otherwise.

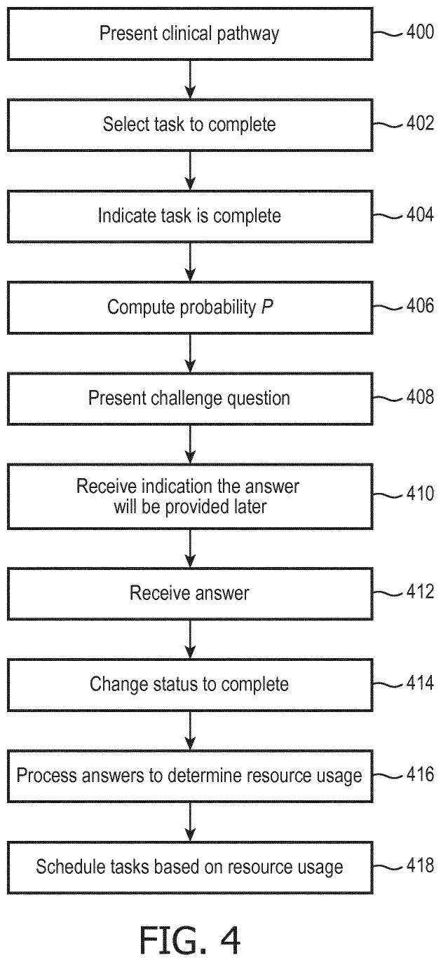

FIG. 4 schematically illustrates a method for capturing information on resource usage in a clinical pathway, where a challenge question is answered within a predetermined time period after completion of a task, but not at the time the CPIS is updated to reflect the task has been completed.

It is to be appreciated that the following is not limiting. That is, one or more acts can be added and/or omitted. Further, an act can occur in a different ordering.

At 400, the computing system 202 executes the instructions of the CPIS module 208, which causes the processor 204 to present at least one clinical pathway of a patient via the output device(s) 224.

In this example, the presented clinical pathway includes multiple tasks, including at least one task identified as a task to be completed.

At 402, the computing system 202, via the input device(s) 222, receives a first signal which selects the clinical task to complete.

At 404, the computing system 202, via the input device(s) 222, receives a second signal which indicates the clinical task has been completed.

At 406, the processor 204 runs the probabilistic algorithm 220 to determine whether and which challenge question will be presented, as disclosed herein and/or otherwise.

At 408, if it is determined that a challenge question will be presented, the processor 204 presents, via the output device(s) 224, a challenge question.

At 410, the computing system 202, via the input device(s) 222, receives a third signal which indicates the challenge question will be answered later.

At 412, the computing system 202, via the input device, receives a fourth signal which answers the challenge question.

At 414, the processor 204, in response to receiving the third signal, changes the status of the task in the clinical pathway to indicate the task is completed.

At 416, the processor 204 stores the answer to the challenge question in the answers 214.

At 418, the processor 204 utilizes the stored answer along with other stored answers for other tasks, clinical pathways, users, etc. to determine resource usage for each task.

At 420, the processor 204 employs the answer analysis algorithm to analyze the resource usage, as disclosed herein and/or otherwise.

At 422, the processor 204 invokes the task scheduler 226 schedule tasks based on the analysis, as disclosed herein and/or otherwise.

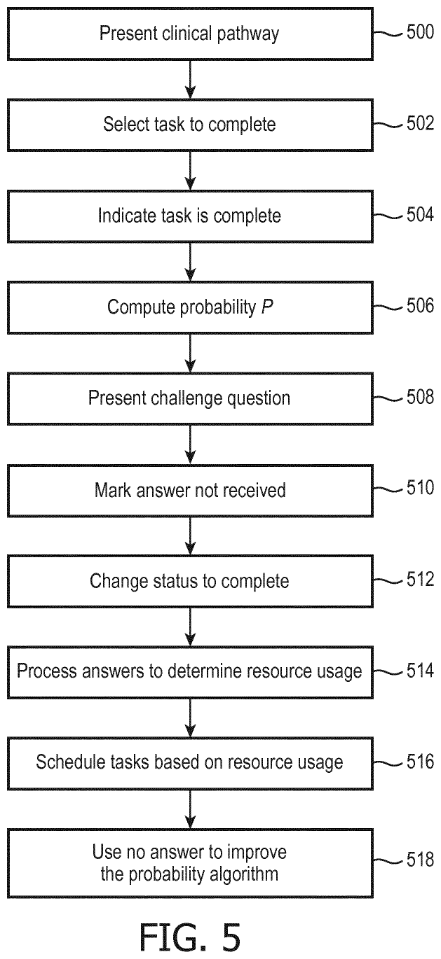

FIG. 5 schematically illustrates a method for capturing information on resource usage in a clinical pathway, where a challenge question is not answered within a predetermined time period after the CPIS is updated to reflect the task has been completed.

It is to be appreciated that the following is not limiting. That is, one or more acts can be added and/or omitted. Further, an act can occur in a different ordering.

At 500, the computing system 202 executes the instructions of the CPIS module 208, which causes the processor 204 to present at least one clinical pathway of a patient via the output device(s) 224.

In this example, the presented clinical pathway includes multiple tasks, including at least one task identified as a task to be completed.

At 502, the computing system 202, via the input device(s) 222, receives a first signal which selects the clinical task to complete.

At 504, the computing system 202, via the input device(s) 222, receives a second signal which indicates the clinical task has been completed.

At 506, the processor 204 runs the probabilistic algorithm 220 to determine whether and which challenge question will be presented, as disclosed herein and/or otherwise.

At 508, if it is determined that a challenge question will be presented, the processor 204 presents, via the output device(s) 224, a challenge question.

At 510, the computing system 202, via the input device, receives a third signal which indicates the challenge question will be answered later.

At 512, the computing system 202, after the lapse of a predetermined time duration from the third signal, records the answer as “failed.”

At 514, the processor 204, in response to recording the answer as “failed,” changes the status of the task in the clinical pathway to indicate the task is completed.

At 516, the processor 204 also stores “failed” in the answers 214.

At 518, the processor 204 utilizes the stored answer along with other stored answers for other tasks, clinical pathways, users, etc. to determine resource usage for each task.

At 520, the processor 204 employs the answer analysis algorithm to analyze the resource usage, as disclosed herein and/or otherwise.

At 522, the processor 204, using the “failed” challenges, improves the information on the response of the users and create a behavior pattern to improve the probability algorithm for showing the challenge. For example, the algorithm may choose to avoid sending challenges at certain hours of the day to minimize “failed” challenges.

At 524, the processor 204 invokes the task scheduler 226 schedule tasks based on the analysis, as disclosed herein and/or otherwise.

The methods herein may be implemented by way of computer readable instructions, encoded or embedded on computer readable storage medium, which, when executed by a computer processor(s), cause the processor(s) to carry out the described acts. Additionally, or alternatively, at least one of the computer readable instructions is carried by a signal, carrier wave or other transitory medium.

The invention has been described herein with reference to the various embodiments. Modifications and alterations may occur to others upon reading the description herein. It is intended that the invention is construed as including all such modifications and alterations insofar as they come within the scope of the appended claims or the equivalents thereof.

1. A system, comprising:
an output device;
input device;
a memory device configured to store instructions and a probabilistic algorithm,
a processor configured to, based on the instruction, to:
display, with the output device, a clinical pathway for a subject, wherein the clinical pathway is in an electronic document with updatable fields;
receive, with the input device, a first signal which selects a first task of the clinical pathway to complete, wherein the first task is not completed;
receive, with the input device, a second signal that which indicates the first task is completed, wherein the first task is completed;
retrieve the probabilistic algorithm from the memory
employ, the probabilistic algorithm to, compute a first probability value for the first task;
determining whether the first probability satisfies a first predetermined threshold:
present, a first challenge question only in response to the first probability value satisfying the first predetermined threshold;
receive, with the input device, a third signal that includes a first answer to the first challenge question;
store the first answer to the first challenge question in the memory; and
update, a first updatable in the electronic document to indicate the first task of the clinical pathway is completed in response to receiving the answer.

2. The system of claim 1, wherein the processor:
analyzes the first answer along with previously received answers with an answer analysis algorithm to determine resource usage for each clinical pathway task.

3. The system of claim 2, wherein the processor:
executes a task scheduler to schedule tasks of clinical pathways based on the determine resource usage.

4. The system of claim 1, wherein the processor:
receive a fourth signal which selects a second task of the clinical pathway to complete, wherein the second task is not completed;
receive a fifth signal that which indicates the second task is completed, wherein the second task is completed;
employ the probabilistic algorithm to compute a second probability value for the second task;
present a second challenge question only in response to the second probability value satisfying a second predetermined threshold;
receive a sixth signal that indicates a second answer to the second challenge question will be provide later;
receive a seventh signal that includes the second answer;
store the second answer in the memory; and
update a second updatable in the electronic document to indicate the second task of the clinical pathway is completed in response to receiving the second answer.

5. The system of claim 4, wherein the processor:
analyzes the first answer, the second answer, and other previously received answers with an answer analysis algorithm to determine resource usage for each clinical pathway task.

6. The system of claim 5, wherein the processor:
executes a task scheduler to schedule tasks of clinical pathways based on the determine resource usage.

7. The system of claim 1, wherein the processor:
receive a fourth signal which selects a second task of the clinical pathway to complete, wherein the second task is not completed;
receive a fifth signal that which indicates the second task is completed, wherein the second task is completed;
employ the probabilistic algorithm to compute a second probability value for the second task;
present a second challenge question only in response to the second probability value satisfying a second predetermined threshold;
receive a sixth signal that indicates a second answer to the second challenge question will be provide later;
record data indicating the second answer was never received in response to not receiving the second answer after lapse of a predetermined time period; and
update a second updatable in the electronic document to indicate the second task of the clinical pathway is completed in response to receiving the second answer.

8. The system of claim 7 wherein the processor:
analyzes the first answer and other previously received answers with an answer analysis algorithm to determine resource usage for each clinical pathway task.

9. The system of claim 8, wherein the processor:
executes a task scheduler to schedule tasks of clinical pathways based on the determine resource usage.

10. The system of claim 7, wherein the processor:
employ the data indicating the second answer was never received to improve the probability algorithm.

11. The system of claim 1, wherein the challenge question is based on one or more criterion from a group consisting of: a Task Identification (ID); a Role ID; a Time of User (ToU); a Resource ID; a Time of Resource (ToR), and an Amount of Resource (AoR).

12. The system of claim 1, wherein the probabilistic algorithm computes probability values with a random number generator to compute a random number that follows a probability distribution in a random manner.

13. The system of claim 1, wherein the probabilistic algorithm computes probability values based on one or more criterion from a group consisting of: a Task ID; a the Role ID; a Frequency of User Input (FoUI); a Frequency of User Access (FoUA); a Time to Last Input (TLI); a “FAIL” Challenge by a User (FCU); a Total Amount of Challenges (TAC); a “DONE” Task by a User (DTU); a Number of Users in a Task (NoUT); a Time of Challenge (ToC); an Average Time of Tasks (AToT); 11) Average Time of Access (AToA); a Task Critical Indicator (TCI); and a Next Task Critical Indicator (NTCI).

14. The system of claim 13, wherein the probabilistic algorithm computes probability values based on a combination of the criterion using one or more factors from a group consisting of: a user is not presented with a challenge every time they use the system; challenges are not presented all the time to a same user; there is a variety of users responding to the challenges; the challenge is presented to more than one user in tasks where there are more than one professionals involved, but not presented to all users in case there are more than two users in play (NoUT); the user does not spend more time completing the task than is normal AToT and/or AToA; a user with FCU close to TAC and/or DTU should not be presented with challenges as often as other users, and a user is not presented with a challenge in case the NTCI exceeds a predetermined threshold and/or is higher than TCI by a predetermined amount.

15. A method, comprising:
displaying, with an output device, a clinical pathway for a subject, wherein the clinical pathway is in an electronic document with updatable fields;
receiving, with an input device, a first signal which selects a first task of the clinical pathway to complete, wherein the first task is not completed;
receiving, with the input device, a second signal that which indicates the first task is completed, wherein the first task is completed;
employing, with a processor, a probabilistic algorithm, which computes a first probability value for the first task;
presenting, with the processor, a first challenge question only in response to the first probability value satisfying a first predetermined threshold;
receiving, with the input device, a third signal that includes a first answer to the first challenge question;
storing the first answer to the first challenge question in a memory;
updating, with the processor, a first updatable in the electronic document to indicate the first task of the clinical pathway is completed in response to receiving the answer;
analyzing the first answer along with previously received answers with an answer analysis algorithm to determine resource usage for each clinical pathway task; and
employing a task scheduler to schedule tasks of clinical pathways based on the determine resource usage.

16. The method of claim 15, further comprising:
receiving a fourth signal which selects a second task of the clinical pathway to complete, wherein the second task is not completed;
receiving a fifth signal that which indicates the second task is completed, wherein the second task is completed;
employing the probabilistic algorithm to compute a second probability value for the second task;
presenting a second challenge question only in response to the second probability value satisfying a second predetermined threshold;
receiving a sixth signal that indicates a second answer to the second challenge question will be provide later;
receiving a seventh signal that includes the second answer;
storing the second answer in the memory;
updating a second updatable in the electronic document to indicate the second task of the clinical pathway is completed in response to receiving the second answer;
analyzing the first answer, the second answer, and other previously received answers with an answer analysis algorithm to determine resource usage for each clinical pathway task; and
employing the task scheduler to schedule tasks of clinical pathways based on the determine resource usage.

17. The method of claim 15, further comprising:
receiving a fourth signal which selects a second task of the clinical pathway to complete, wherein the second task is not completed;
receiving a fifth signal that which indicates the second task is completed, wherein the second task is completed;
employing the probabilistic algorithm to compute a second probability value for the second task;
presenting a second challenge question only in response to the second probability value satisfying a second predetermined threshold;
receiving a sixth signal that indicates a second answer to the second challenge question will be provide later;
recording data indicating the second answer was never received in response to not receiving the second answer after lapse of a predetermined time period;
updating a second updatable in the electronic document to indicate the second task of the clinical pathway is completed in response to receiving the second answer;
analyzing the first answer and other previously received answers with an answer analysis algorithm to determine resource usage for each clinical pathway task; and
employing the task scheduler to schedule tasks of clinical pathways based on the determine resource usage.

18. The method of claim 16, further comprising:
employing the data indicating the second answer was never received to improve the probability algorithm.

19. The method of claim 16, further comprising:
computing a random number as the probability value.

20. A non-transitory computer readable medium encoded with computer executable instructions, which, when executed by a processor of a computer, cause the processor to:
display a clinical pathway for a subject, wherein the clinical pathway is in an electronic document with updatable fields;
receive a first signal which selects a task of the clinical pathway to complete, wherein the task is not completed;
receive a second signal that which indicates the task is completed, wherein the task is completed;
employ a probabilistic algorithm, which computes a probability value for the task;
present a challenge question only in response to the probability value satisfying a predetermined threshold;
receive a third signal that includes an answer to the challenge question;
store the answer to the challenge question in a memory;
update an updatable in the electronic document to indicate the task of the clinical pathway is completed in response to receiving the answer;
analyze the answer along with previously received answers with an answer analysis algorithm to determine resource usage for each clinical pathway task; and
employ a task scheduler to schedule tasks of clinical pathways based on the determine resource usage.
